(19) United States
(12) Patent Application Publication
(10) Pub. No.: US 2026/0108149 A1
(43) Pub. Date: Apr. 23, 2026

(54) APPARATUS WITH SWITCHABLE CONFIGURATION FOR OPTICAL COHERENCE TOMOGRAPHY AND METROLOGY OF AN EYE

(71) Applicant: Alcon Inc., Fribourg (CH)

(72) Inventors: Steven James Frisken, Vaucluse (AU); Armin Georg Segref, Mulgrave (AU)

(21) Appl. No.: 19/365,614

(22) Filed: Oct. 22, 2025

(30) Foreign Application Priority Data
Oct. 23, 2024 (AU) 2024903434

(51) Int. Cl. A61B 3/10; A61B 3/107; A61B 3/117; A61B 3/12; A61B 3/14; G16H 40/63

(52) U.S. Cl. CPC A61B 3/102; A61B 3/1005; A61B 3/1015; A61B 3/107; A61B 3/117; A61B 3/1225; A61B 3/145; G16H 40/63

(57) ABSTRACT

Apparatus and methods are presented for optical coherence tomography and metrology of an eye, with the apparatus being switchable between anterior chamber and posterior chamber modes. In the posterior chamber mode the apparatus is configured to provide tomographic imaging of the retina, while in the anterior chamber mode the apparatus is configured to provide tomographic imaging of one or more features in the anterior chamber and one or more additional imaging or metrology modalities including at least one of wavefront sensing and reflection based corneal topography. In preferred embodiments the apparatus comprises an optical relay having one or more electrically actuatable elements for switching the apparatus between the anterior chamber and posterior chamber modes.

### FIELD OF THE INVENTION

The invention relates to apparatus and methods for optical coherence tomography and metrology of an eye, in particular with a switchable configuration for imaging the anterior chamber in a first mode and the retina in a second mode. However it will be appreciated that the invention is not limited to this particular field of use.

### BACKGROUND OF THE INVENTION

Any discussion of the prior art throughout the specification should in no way be considered as an admission that such prior art is widely known or forms part of the common general knowledge in the field.

Efficient workflow is critical in an ophthalmic or optometry setting. Having to perform multiple measurements on a patient's eyes with different instruments is inconvenient for the patient and presents space and workflow challenges in a busy environment. The need to have several different instruments is also expensive. Additionally, accuracy is essential for measurements that are relied on for critical procedures such as refractive or cataract surgery, so having independent redundant means for measurement of the critical parameters can provide a higher level of confidence in the outcomes. For example, corneal topography could be measured with a camera-based technique such as Scheimpflug imaging as well as with a reflective topography technique such as narrow cone or Placido disc, and the results compared to identify potential measurement complications such as tear film instability.

More recently, volume-based optical coherence tomography (OCT) measurements have been able to provide detailed volume structure and metrology of the eye including of the posterior corneal surface, corneal thickness, lens geometry and tilt and the retina. A volume-based OCT instrument capable of in-vivo measurement of structures in the anterior and posterior chambers of the eye has been described in published US patent application No 2019/0365220 A1. It would be advantageous to enhance this instrument with other modalities such as wavefront sensing, videography in the visible or NIR and tear film reflection measurement for analysis of tear film topography or stability. It is, however, not straightforward to integrate these additional modalities into such an OCT apparatus, due to the existing complexity of the optical train, the sensitivity of OCT and wavefront measurements to back reflections and the difficulty of incorporating a Placido disc or other distributed source within a wide-field telecentric OCT system in a way that also allows the instrument to approach the eye with a suitable working distance. In addition, any OCT system configured to switch between retinal and anterior chamber modalities must be able to provide an adequate working distance for both modalities, path length matching with a reference beam and a mechanism for adjusting the focusing of retinal beams onto or near the retina to account for different eye types or prescriptions.

Unless the context clearly requires otherwise, throughout the description and the claims the words ‘comprising’, ‘comprises’ and the like are to be construed in an inclusive sense as opposed to an exclusive or exhaustive sense. That is, they are to be construed in the sense of ‘including, but not limited to’. Similarly, unless the context clearly requires otherwise, the word ‘or’ is to be construed in an inclusive sense rather than an exhaustive sense. That is, the expression ‘A or B’ is to be construed as meaning ‘A, or B, or both A and B’.

### OBJECT OF THE INVENTION

It is an object of the present invention to overcome or ameliorate at least one of the limitations of the prior art, or to provide a useful alternative. It is an object of the present invention in a preferred form to provide an OCT apparatus suitable for imaging the anterior chamber and retina of an eye and with one or more additional imaging or metrology modalities.

### SUMMARY OF THE INVENTION

According to a first aspect of the present invention there is provided an apparatus for optical coherence tomography and metrology of an eye, the apparatus being switchable between an anterior chamber mode and a posterior chamber mode, wherein in the anterior chamber mode the apparatus is configured to provide:
tomographic imaging including metrology of one or more of corneal thickness, anterior chamber depth, lens thickness and axial length of the eye;
wavefront sensing measurements; and
metrology of one or more optical or biological properties selected from the group comprising reflection based corneal topography, tear film breakup and en-face photography or videography;
and wherein in the posterior chamber mode the apparatus is configured to provide tomographic imaging of the retina of the eye.

According to a second aspect of the present invention there is provided an apparatus for optical coherence tomography and metrology of an eye, the apparatus being switchable between an anterior chamber mode and a posterior chamber mode, wherein in the anterior chamber mode the apparatus is configured to provide:
tomographic imaging including metrology of one or more of corneal thickness, anterior chamber depth, lens thickness and axial length of the eye;
reflection based corneal topography; and
metrology of one or more optical or biological properties selected from the group comprising wavefront sensing measurements, tear film breakup and en-face photography or videography;
and wherein in the posterior chamber mode the apparatus is configured to provide tomographic imaging of the retina of the eye.

The apparatus according to the first and second aspects share several preferments.

Preferably, the apparatus comprises an optical relay having one or more electrically actuatable elements for switching the apparatus between the anterior chamber mode and the posterior chamber mode. The one or more electrically actuatable elements preferably comprise a movable element of a zoom lens.

The zoom lens preferably has a focal length in the range of 30 to 150 mm, more preferably in the range of 60 to 80 mm and most preferably around 72 mm when the apparatus is in the anterior chamber mode. The zoom lens preferably has a focal length in the range of 15 to 75 mm, more preferably in the range of 30 to 40 mm and most preferably around 36 mm when the apparatus is in the posterior chamber mode. In preferred embodiments the optical relay includes a lens assembly having a fixed focal length in the range of 30 to 150 mm, more preferably in the range of 60 to 80 mm and most preferably around 72 mm.

Preferably, the one or more electrically actuatable elements comprises one or more lenses for insertion into the optical path when switching to the posterior chamber mode. In preferred embodiments the one or more lenses comprises a lens pair. The separation between the lenses of the lens pair is preferably adjustable to suit the prescription of the eye.

In certain embodiments the apparatus comprises manually interchangeable components for switching the apparatus between the anterior chamber mode and the posterior chamber mode, wherein the manually interchangeable components have electrical interfaces that connect to the apparatus at a registration point. The registration point is preferably achieved with a magnetic ramp and lock. In certain embodiment the apparatus comprises means for identifying the manually interchangeable component currently in use. The apparatus preferably comprises stored calibration data for each of the manually interchangeable components.

The apparatus according to the first aspect is preferably configured to provide reflection based corneal topography when in the anterior chamber mode. Preferably, the apparatus is configured to provide metrology of tear film breakup and en-face photography or videography when in the anterior chamber mode.

The apparatus according to the second aspect is preferably configured to provide wavefront sensing measurements when in the anterior chamber mode. Preferably, the apparatus is configured to provide metrology of tear film breakup and en-face photography or videography when in the anterior chamber mode.

According to a third aspect of the present invention there is provided an article of manufacture comprising a non-transitory computer usable medium having a computer readable program code configured to operate the apparatus according to the first or second aspect.

### BRIEF DESCRIPTION OF THE DRAWINGS

Preferred embodiments of the invention will now be described, by way of example only, with reference to the accompanying drawings in which:

FIG. 1 illustrates in schematic plan view an OCT apparatus suitable for in vivo ‘snapshot’ tomographic imaging of various structures in an eye;

FIG. 2A shows in schematic plan view a dual configuration optical relay suitable for use in an OCT apparatus, configured for tomographic imaging of the anterior chamber of an eye;

FIG. 2B shows in schematic plan view the dual configuration optical relay of FIG. 2A in a second configuration, suitable for tomographic imaging of the retina of an eye;

FIG. 2C depicts a lens pair component that can be moved into the optical train for the retinal imaging configuration shown in FIG. 2B;

FIG. 3 shows in schematic plan view a fixation target and videography module that can be multiplexed into the dual configuration optical relay shown in FIGS. 2A and 2B;

FIG. 4 shows in schematic form the addition of a tear film/corneal topography module to an OCT apparatus having the dual configuration optical relay shown in FIGS. 2A and 2B; and

FIG. 5 shows in schematic plan view a wavefront sensor module that can be multiplexed into the dual configuration optical relay shown in FIGS. 2A and 2B.

### DETAILED DESCRIPTION OF THE INVENTION

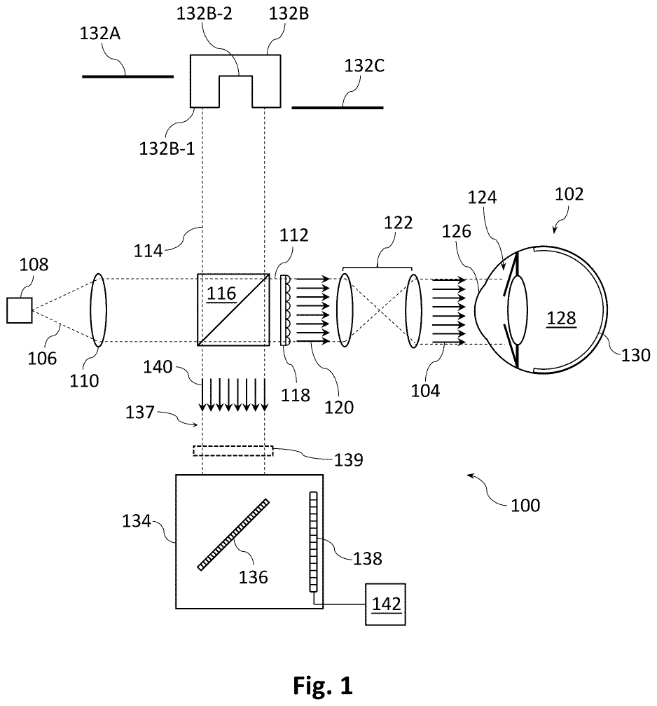

FIG. 1 shows in schematic plan view an optical coherence tomography (OCT) apparatus 100 suitable for in vivo ‘snapshot’ tomographic imaging of various structures in a sample eye 102 using a grid of beamlets 104. As described in more detail in published US patent application Nos 2019/0365220 A1 and 2021/0244278 A1, the contents of which are incorporated herein by reference, light 106 from a broadband source 108 such as a superluminescent diode with centre wavelength 840 nm and bandwidth of 40 nm is collimated by a collimating element 110 such as a lens or parabolic mirror then split into sample and reference beams 112, 114 with a beam splitting cube 116. The sample arm includes a spatial sampling element 118 in the form of a two-dimensional (2-D) lenslet array for generating from the sample beam 112 a 2-D array of beamlets 120 that are relayed to the eye 102 via a relay 122 of optical power elements, typically lenses but possibly including one or more mirrors. The form of the array of beamlets 104 emerging from the relay 122, e.g. parallel or converging, is determined by the design of the relay 122, and the OCT apparatus 100 may have two or more manually interchangeable relays 122 designed to project the beamlets 104 onto one or more structures in the anterior chamber 124, such as the cornea 126, or in the posterior chamber 128, such as the retina 130, or both.

The reference arm typically has a number of interchangeable mirrors 132A, 132B, 132C for optical path length matching to structures of interest in the eye 102. In preferred embodiments the range of reference arm mirrors includes a compound mirror 132B having axially and laterally separated reflective surfaces 132B-1 and 132B-2. As explained in US 2019/0365229 A1 this facilitates simultaneous acquisition of data from the anterior chamber 124 and the retina 130, e.g. for measuring axial length while imaging the anterior chamber. In preferred embodiments the beam splitting cube 116 is a polarisation beam splitter and the sample and reference arms include quarter wave plates (not shown) for efficient use of light 106 from the broadband source 108. In certain embodiments the quarter wave plate in the sample arm is rotatable to provide the OCT apparatus 100 with polarisation sensitivity as described in PCT patent application No PCT/AU2024/050377 entitled ‘Apparatus and method for polarisation-sensitive optical coherence tomography’, the contents of which are incorporated herein by reference.

Light scattered or reflected from various structures of the eye 102 passes back through the relay 122, then is refocused by the lenslet array 118, mixed with the reference beam 114 at the beam splitting cube 116 and directed into a spectrometer 134 comprising a wavelength dispersive element 136 and a 2-D sensor array 138. If the beam splitting cube 116 is a polarisation beam splitter the detector arm 137 may include a polarisation analyser 139 such as a linear polariser to interfere the light in the reference beam 114 and returning sample beamlets 140. Portions of the interferogram corresponding to the returning beamlets 140 mixed with the reference beam 114 can be dispersed onto separate sets of pixels of the 2-D sensor array 138, for read-out in a single frame followed by analysis in a computer 142 equipped with suitable computer readable program code. This provides simultaneous or ‘snapshot’ acquisition of tomographic data from a plurality of points across the eye 102 accessed by the beamlets 104. A series of snapshot acquisitions with the beamlet grid 104 translated in one or two dimensions to fill in the on-eye gaps between the beamlets enables the computation of a complete tomographic image of one or more selected structures in the eye 102.

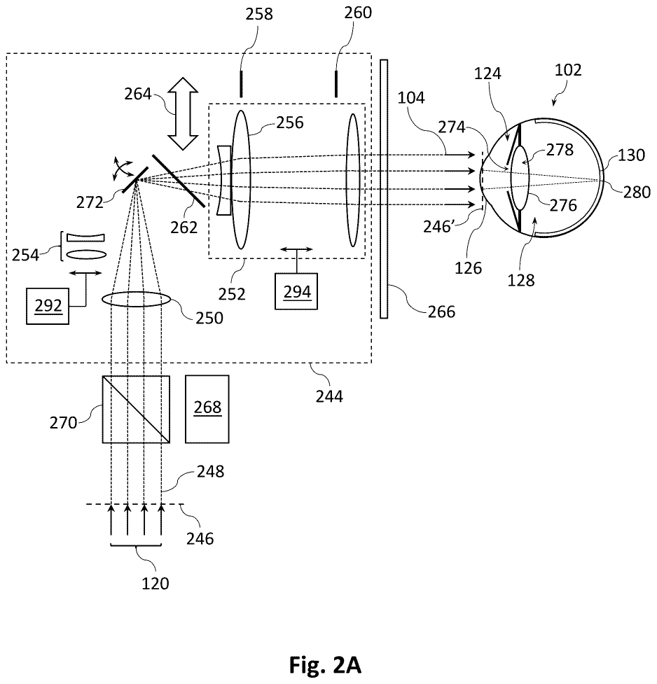

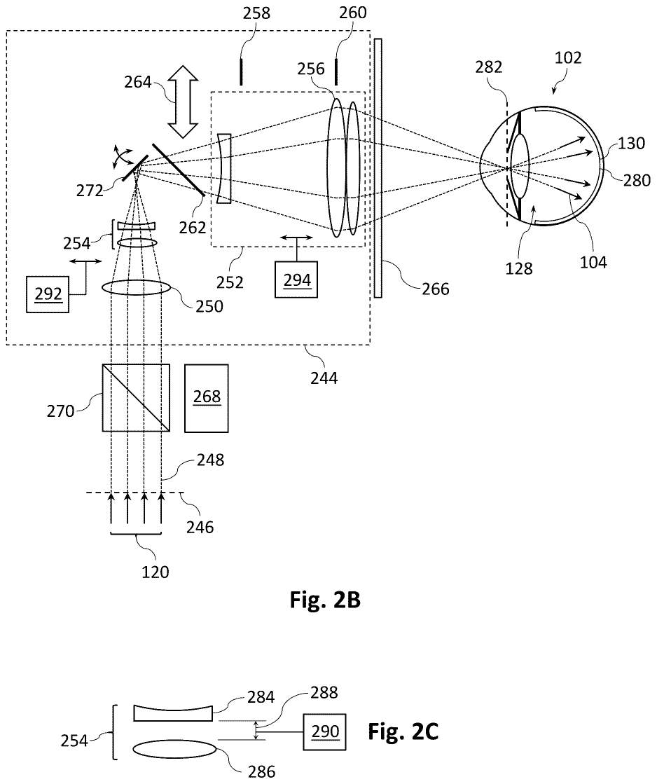

FIG. 2A shows in schematic plan view a dual configuration optical relay 244 according to a first embodiment of the present invention, suitable for use in an OCT and metrology apparatus that can, depending on the particular configuration of the relay 244 and the presence of additional components, provide tomographic data from one or more structures in the anterior chamber 124, tomographic data from one or more structures in the posterior chamber 128 and biometric measurements such as wavefront sensing, corneal topography, tear film breakup or en-face photography or videography.

Incident on the dual configuration optical relay 244 is a 2-D array of beamlets 120 generated using an (840±40) nm broadband source 108 and a 2-D lenslet array 118 as described previously with reference to FIG. 1, with the relay 244 standing in place of the simple relay 122 depicted in FIG. 1. In a preferred embodiment the beamlets 120 propagate in parallel as shown and occupy a grid of locations with a spacing of 400 μm on a square or rectangular lattice comprising 42 horizontal locations and 24 vertical locations, with beam waists of approximately 30 μm at a focal surface 246 of the lenslet array 118 (not shown in FIG. 2A). In other embodiments the array of beamlets 120 may be converging or diverging, while the beamlet waists may be provided along a planar surface 246 as shown or along a curved surface e.g. for optimal sampling of the cornea 126 or other curved structure in the anterior chamber 124 of an eye 102. The paths of the beamlets 120 through the relay 244 to the eye 102 are represented by their centre rays 248.

The relay 244 comprises a first lens assembly 250 and a second lens assembly 252. The relay 244 may also comprise one or more lenses such as a lens pair 254 that can be moved in and out of optical path or train, i.e. the path of the beamlets 120, using an actuator 292. The lens pair 254 is described in more detail with reference to FIG. 2C. For simplicity the first lens assembly 250 is depicted as a single lens, but in preferred embodiments is a multi-element lens assembly designed to minimise wavefront distortion and provide telecentricity and minimal distortion of the beamlet grid when combined with the second lens assembly 252 in a 4F configuration. Preferably, the focal surface 246 is located at the focal plane of the first lens assembly 250. The second lens assembly 252 is a zoom lens that includes a movable lens 256 that can be moved between first and second stop positions 258, 260 using an actuator 294. In a particularly preferred embodiment the first lens assembly 250 has a fixed focal length of around 72 mm, while the second lens assembly 252 has a focal length of around 72 mm when the movable lens 256 is at the first stop position 258 for the anterior chamber mode or around 36 mm when the movable lens is at the second stop position 260 for the posterior chamber mode. These particular focal lengths are chosen to provide a convenient working distance between the eye 102 and an OCT/metrology apparatus incorporating the optical relay 244. In other embodiments the focal length of the first lens assembly 250 is in the range of 30 to 150 mm, more preferably 60 to 80 mm, while the focal length of the second lens assembly 252 is in the range of 30 to 150 mm, more preferably 60 to 80 mm, when the movable lens 256 is at the first stop position 258, or in the range of 15 to 75 mm, more preferably 30 to 40 mm, when the movable lens is at the second stop position 260.

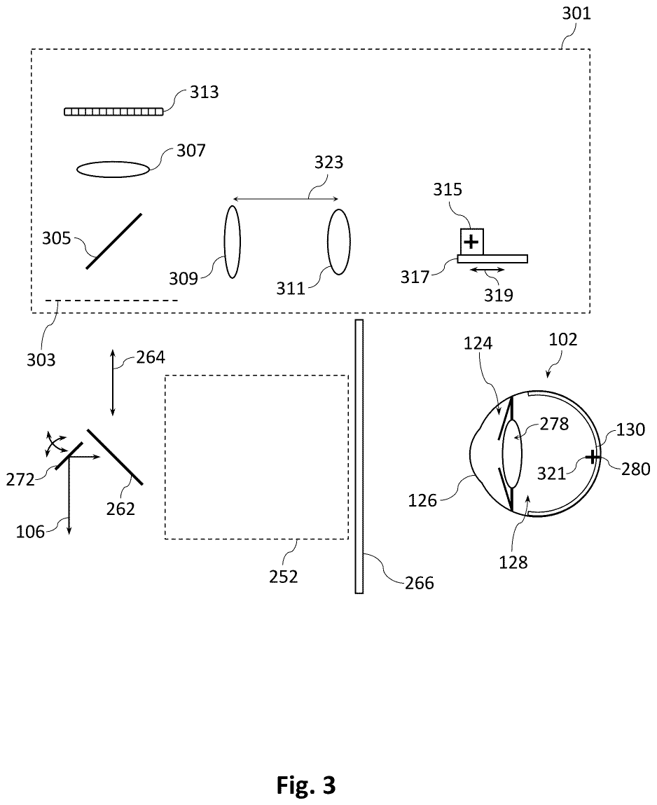

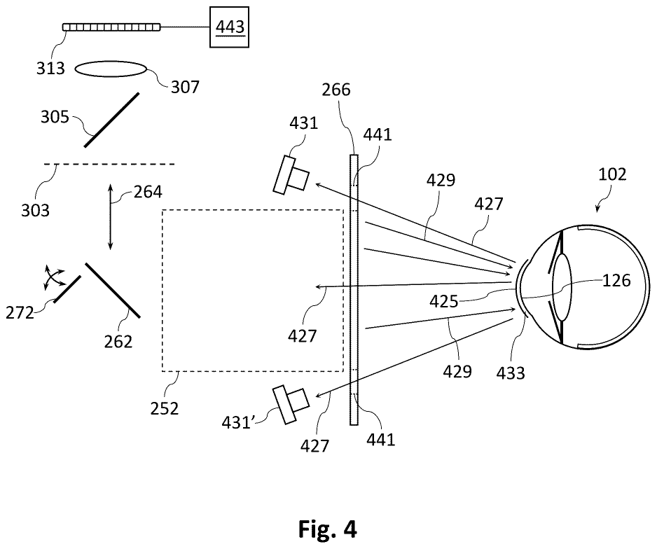

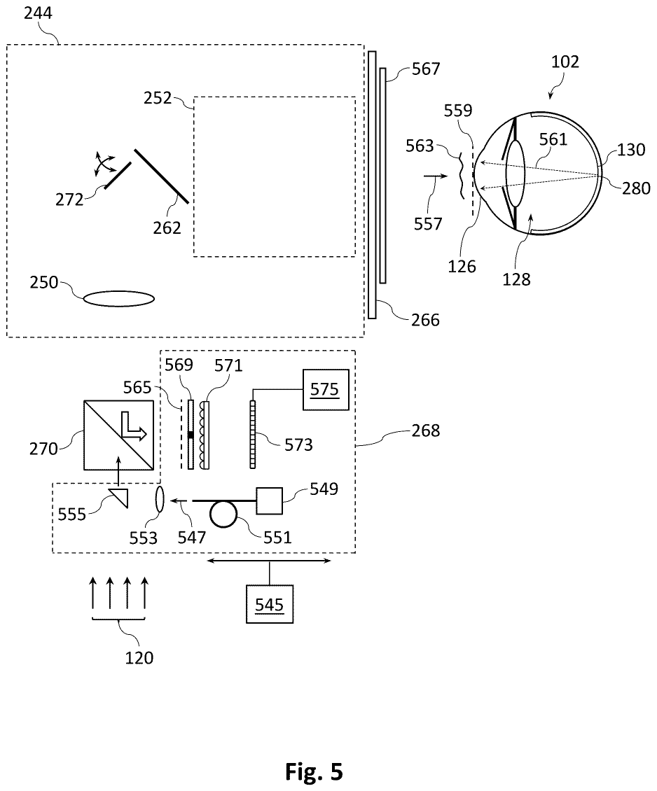

FIG. 2A also shows several other components for providing additional biometric imaging or metrology functions. These include: a dichroic beam splitter 262 for multiplexing in light 264 of a different wavelength range, e.g. in the visible, for fixation targets or focal plane array videography or photography, to be described with reference to FIGS. 3 and 4; a scattering disc 266 for creating an illumination object for measurement of corneal topography or tear film break-up, to be described with reference to FIG. 4; and a wavefront sensor 268 multiplexed into the optical path with a polarisation beam splitter (PBS) 270, to be described with reference to FIG. 5. FIG. 2A also shows a 2-D beam-steering element 272 such as an electromagnetically or electrostatically actuated mirror or beam deflector for dithering or scanning the relayed array of beamlets 104 across the eye 102.

As depicted in FIG. 2A with the lens pair 254 out of the optical train and the movable lens 256 at the first stop position 258, the dual configuration optical relay 244 is configured for tomographic imaging of the anterior chamber 124 of the sample eye 102, generating an array of beamlets 104 with waists at a focal surface 246′ conjugate to the focal surface 246 and proximate to or within the anterior chamber. In this configuration the first and second lens assemblies 250, 252 form a non-magnifying 4F relay system, with the incoming array of beamlets 120 converged by the first lens assembly 250 onto the beam steering element 272. This provides an expanded beamlet size of approximately 3 mm at the back focal plane of the first lens assembly 250, coincident with the beam steering element 272. The second lens assembly 252 has its principal plane located one focal length from the beam steering element 272 to re-form an array of beamlets 104 propagating in parallel towards the eye 102. Light from the beamlets 104 scattered or reflected from various structures of the eye 102 passes back through the relay 244 for spectral analysis and computation of a tomographic image as described previously with reference to FIG. 1. We note that some of the more central beamlets in the array 104 will pass through the pupil 274 and be focused onto the retina 130 by the optical power elements of the eye 102, enabling measurement of the axial length of the eye while other beamlets probe the anterior chamber 124.

An OCT and metrology apparatus equipped with a dual configuration optical relay 244 in the ‘anterior chamber’ configuration shown in FIG. 2A can provide tomographic data from the anterior chamber 124, including metrology of one or more of corneal thickness, lens thickness and anterior chamber depth, as well as axial length of the eye 102, plus metrology of one or more optical or biological properties of the eye selected from the group comprising wavefront sensing, corneal topography, tear film breakup and en-face photography or videography. Anterior chamber depth may be measured from the corneal apex to the posterior surface 276 of the lens 278, while axial length may be measured from the corneal apex to the fovea 280.

FIG. 2B shows in schematic plan view the dual configuration optical relay 244 in a second, ‘posterior chamber’ configuration, suitable for tomographic imaging of the retina 130. In this configuration the movable lens 256 of the second lens assembly 252 is at the second stop position 260 and the lens pair 254 is moved into the optical train. The shift in angular overlap position of the beamlets 120 caused by the lens pair 254, in combination with the shorter focal length of the second lens assembly 252 and the optical power elements of the eye 102, causes the overlapping expanded beamlets to be reimaged with minimal magnification to a location proximate to the pupil plane 282, then mapped onto a grid of probe beamlets 104 incident on the retina 130. Light from the probe beamlets 104 scattered or reflected from various layers of the retina 130 passes back through the relay 244 for spectral analysis and computation of a tomographic image as described previously with reference to FIG. 1.

When the dual configuration optical relay 244 is switched between its ‘anterior chamber’ and ‘posterior chamber’ configurations, the optical path length of the reference arm can be adjusted as required, e.g. by actuated selection of a suitable reference mirror 132A, 132B or 132C as described previously with reference to FIG. 1.

FIG. 2C shows a more detailed view of the lens pair 254 that may be moved into the optical train for the ‘posterior chamber’ configuration of the dual configuration optical relay 244 to increase the angular spread of beamlets 104 on the retina 130. The lens pair 254 includes a concave lens 284 and a convex lens 286 with an adjustable separation 288 controlled by an electrical actuator 290. Control of the separation 288 allows the optical relay 244, and the OCT apparatus as a whole, to be adjusted to suit the prescription of the eye 102 under test.

Preferably, the various actuators in the dual configuration optical relay 244 are software-initiated. This includes the actuators 292, 294 and 290 used to move the lens pair 254 in and out of the optical train, move the lens 256 between stop positions in the second lens assembly 252 and adjust the separation of the lens pair 254, as well as any actuators used to select the appropriate reference mirror.

FIG. 3 shows in schematic plan view a fixation target and videography module 301 that can be multiplexed into the dual configuration optical relay 244 of FIGS. 2A and 2B by use of a dichroic beam splitter 262. In a preferred embodiment the dichroic beam splitter 262 is designed to transmit the (840±40) nm light 106 used for the tomographic imaging and reflect light 264 in the visible and 940 nm regions used for videography, fixation targets and reflection based corneal topography. Ideally the reflection of the dichroic beam splitter 262 will be near 0% around 840 nm and near 100% in the remainder of the visible and near infrared regions, but in reality due to complexities of multilayer dielectric designs may be <10 % at the OCT signal band around 840 nm and >70% in the remainder of the visible and near infrared regions. In preferred embodiments the dichroic beam splitter 262 is designed to be substantially polarisation insensitive in both amplitude and phase, to minimise perturbations of the transmitted or reflected light.

We consider firstly the case of the videography and on-axis tear film reflection when the dual configuration optical relay 244 is in the first, ‘anterior chamber’, configuration as shown in FIG. 2A, with the focal plane of the second lens assembly 252 lying at a location 303 to which other components of the fixation target and videography module 301 will be referenced. The fixation target and videography module 301 comprises a beam splitter 305 such as a non-polarising beam splitter, a polarising beam splitter or a dichroic filter, lenses or other optical power elements 307, 309 and 311, a 2-D sensor array 313 such as a CMOS camera and a fixation target 315. When the anterior chamber 124 of the eye 102 is illuminated, e.g. by a diffuse light source such as ambient light for videography or a structured scattered illumination from the scattering disc 266, the second lens assembly 252 and lens 307 act in concert to provide telecentric imaging of the anterior chamber, with the lens 307 positioned such that its focal plane is approximately coincident with the focal plane 303 of the second lens assembly. The image formed at the CMOS camera 313 will be demagnified by the ratio f₃₀₇/f₂₅₂ i.e. the ratio of the focal lengths of the lens 307 and lens assembly 252. In one example, with f₃₀₇=10 mm and f₂₅₂=72 mm, the demagnification ratio will be approximately 0.14. A CMOS camera 313 with suitably high resolution and a suitably fast frame rate can be employed for videography, or in a single capture mode for photography.

The fixation target 315, which may for example be a miniature LED display emitting in the visible region, is designed, in combination with the lenses 309 and 311 having focal lengths f₃₀₉ and f₃₁₁, to relay an image in focus to the fovea 280 of the eye 102 so that the patient can keep their eye steady and well positioned during the various measurements. The lens 309 is preferably positioned such that its back focal plane lies at location 303. In the illustrated embodiment the fixation target 315 is mounted on an electrically actuated translation stage 317, enabling the switching between the ‘anterior chamber’ and ‘posterior chamber’ configurations of the dual configuration optical relay 244 to be compensated for by a software-initiated translation 319. In an alternative embodiment the switching of the relay 244 is compensated for by moving a lens into or out of the beam path from the fixation target 315.

When employed in unison with the ‘anterior chamber’ configuration of the dual configuration optical relay 244, the fixation target 315 is positioned at the focal plane of the lens 311 to create, for an emmetropic patient, an image of the fixation target at the plane 303. This image is in turn relayed through the 4F combination of the lens assembly 252 and the cornea 126 and lens 278 of the eye 102 to generate an image 321 of the fixation target 315 on the fovea 280. The module 301 can be adjusted for the prescription of the patient by shifting 319 the fixation target 315 to provide the opposite optical power. When the fixation target 315 is used together with the ‘anterior chamber’ configuration of the optical relay 244 as shown in FIG. 2A the pair of lenses 309 and 311 does not need to be telecentric. In a preferred embodiment we choose to set the distance 323 between these lenses 309 and 311 to be equal to 2*f₃₁₁+f₃₀₉. This allows us to use the same translation stage 319 to move to a second range where the fixation target 315 is now located a distance of about 2*f₃₁₁ away from the lens 311. In this location the far field of the fixation target 315 is now projected onto the plane 303, which is appropriate for when the optical relay 244 is in the ‘posterior chamber’ configuration as shown in FIG. 2B. In this case actuation of the lens assembly 252 to its shorter focal length, say f=36 mm, will image the projected far field of the fixation target 315 to the corneal plane. The focusing action of the cornea 126 and lens 278 will transform the fixation target 315 to a focused image 321 at the fovea 280. Again, adjustment 319 of the fixation target's axial position enables compensation of a non-emmetropic eye to create a focused fixation image 321.

In one particular embodiment, the lenses 309, 311 and their separation 323 are chosen such that f₃₀₉=10 mm, f₃₁₁=5 mm and separation 323=20 mm. The separation 323 could be adjusted by design to provide a different magnification of the fixation target 315 on the fovea 280.

FIG. 4 shows schematically how certain components of the previously described dual configuration optical relay 244 and fixation target and videography module 301 can provide information on the eye's strongest optical power element, the corneal tear film/air interface 425. In particular, reflections 427 from the tear film 433 of light 429 emanating from an illuminated scattering disc 266 can be detected with an en-face camera 313 and optionally one or more off-axis cameras 431, 431′ to provide detailed positional, topographic and quality analysis of the tear film 433.

In preferred embodiments the scattering disc 266 is a transparent glass or polymer plate with one or more light sources injecting light through its circumferential edge and a plurality of laser-machined or etched scattering elements forming for example a Placido disc structure, a dot structure or a spiral structure. In certain embodiments the central portion of the scattering disc 266 and selected other portions 441 have no or few scattering elements to provide a clear path for light reflected 427 from the tear film 433 to be captured by the cameras 313, 431 or 431′. A clear central region of the scattering disc 266 may also provide a clear passage for beamlet light propagating towards and back from the eye 102 for the previously described OCT imaging mode, although some scattering loss can be tolerated.

The reflection images captured by the on-axis camera 313 and optionally by the one or more off-axis cameras 431, 431′ can be analysed in a computer 443, which may be the same as the computer 142 used for spectral analysis of the OCT signal, equipped with suitable computer readable program to provide a topographic map of the tear film 433 or information about the stability or quality of the tear film as is well known in the field of Placido disc topography for example. Generally, because of the curvature of the cornea 126 the different cameras 313, 431, 431′ will capture images of overlapping but offset regions of the tear film 433.

The imaging path to the on-axis camera 313 relies on the separation of light paths via the dichroic beam splitter 262 and a telecentric relay provided by the second lens assembly 252 and the lens 307. With reference to FIG. 2A, the movable lens 256 of the second lens assembly 252 should be able to be set in a well-characterised stop position 258 to provide a highly repeatable and accurate focal length, so that quantitative analysis of the reflection images can provide accurate measurement of corneal curvature over the eye, with knowledge of the relative locations of the scattering elements in the disc 266. Information on the relative location of the scattering elements to the eye 102 can be obtained from the OCT signal or inferred from the relative images on each of the three cameras 313, 431, 431′.

FIG. 5 shows in schematic plan view a wavefront sensor module 268 that in preferred embodiments can be inserted by an actuator 545 into the OCT beam path, as represented by the beamlet array 120, when the dual configuration optical relay 244 is in the ‘anterior chamber’ configuration as shown in FIG. 2A. The purpose of the wavefront sensor module 268 is to provide measurements of ocular aberrations of a sample eye 102 when a small beam of light scatters from the fovea 280.

A probe beam of polarised light 547 generated from a light source 549 such as a SLED or laser is coupled into a polarisation maintaining optical fibre 551, collimated by a collimation element 553 such as a gradient index or spherical lens and directed into the OCT beam path by a micro prism 555 and a polarising beam splitter 270.

The polarised probe beam 547 is relayed by the dual configuration optical relay 244 in its ‘anterior chamber’ configuration to provide a focused beamlet 557 with a diameter of approximately 100 μm at or near the corneal plane 559. The steering mirror 272 may be used to direct the location of the beamlet 557 with respect to the corneal apex so that the specular reflection from the cornea 126 is not captured within the numerical aperture of the relay 244. Alternatively, polarisation discrimination can be used to reject the strong specular reflection. The fixation target 313 shown in FIG. 3, which is coupled into the relay 244 via the dichroic mirror 262 in the visible, ensures that the beamlet 557 is directed onto the central fovea 280. The fixation target can also be used to ensure the eye 102 is in a relaxed state, by providing a sequence of defocused images appropriate for a given patient prescription.

Light 561 scattered from the fovea 280 and exiting the eye 102 will be formed into a wavefront 563 that captures information about the refractive power and aberrations of the eye under test. The wavefront 563 is relayed by the optical relay 244 in its ‘anterior chamber’ configuration to a conjugate plane 565 via the polarisation beam splitter 270. If the sample arm of the OCT apparatus 100 described with reference to FIG. 1 includes a rotatable quarter wave plate 567, in the wavefront sensing function this quarter wave plate can be rotated by 45 degrees from its usual OCT orientation to be parallel to the polarisation state of the incoming beamlet 557. In this case, because light 561 scattered from the retina 130 including the fovea 280 is partially depolarised, it is the depolarised component that will be directed towards the conjugate plane 565. A central obscuration target 569 may remove any remaining specular corneal reflection to improve the signal to noise ratio and a 2-D lenslet array 571 focuses the wavefront to a plurality of spots on a 2D-focal plane array 573. Noting that the positions on the focal plane array 573 of the centroids of each of the generated spots are determined by the local slope of the wavefront 563 at the lenslet array 571, the power and aberrations of the wavefront can be calculated from the detected grid of centroids and calibrated with respect to a reference wavefront using a computer 575 equipped with suitable machine-readable program code, as is well known in the art of Shack-Hartmann wavefront sensing. The computer 575 may be the same as the computer 142 used for spectral analysis of the OCT signal.

In preferred embodiments the wavefront sensor module 268 can be moved in and out of the OCT beam path, e.g. using a software-initiated actuator 545, although the wavefront sensor module may alternatively be fixed. If the wavefront sensor module 268 is movable, it is also preferred for the polarisation beam splitter 270 to be moved in and out of the OCT beam path, especially if the beam splitting cube 116 used to separate the forward and reverse light paths 120, 140 in FIG. 1 is a polarisation beam splitter. On the other hand, for a single polarisation OCT implementation with a non-polarising beam splitting cube 116, the polarisation beam splitter 270 may be fixed. In yet another embodiment, light for the wavefront sensing function can be multiplexed into the OCT beam path on the basis of wavelength using a dichroic mirror, in which case the micro prism 555 may be located between the dichroic mirror and the central obscuration target 569. In this embodiment the OCT beamlets 120 may be in a band around 840 nm and the wavefront sensing probe beam 547 may be around 810 nm.

As described previously with reference to FIGS. 2A and 2B, an OCT apparatus equipped with the dual configuration optical relay 244 transitions between anterior chamber and posterior chamber imaging modes by electrical actuation of a zoom lens, in particular movement of a lens element 256 between first and second stop positions 258, 260. In alternative embodiments the transition can be achieved manually by means of a pair of interchangeable components attached to the main body of the instrument. These interchangeable components may include lens relays suitable for anterior chamber or posterior chamber imaging, as well as wave plates, LEDs and scattering discs. One or both of the interchangeable components may also have an electrical interface for provision of power to electrical elements such as LEDs, mechanical wave plate rotators and focus mechanisms. In certain embodiments the interchangeable components are secured accurately and repeatably in place by a magnetic registration and alignment ramp and lock, which allows for ease of removal by rotation to counter the magnetic force and with electrical interfaces aligned and contacted with the apparatus at the registration point. In certain embodiments the registration point includes a hard stop for rotational and translational location that clicks into place when an interchangeable component is correctly positioned. The apparatus may also be able to use the electrical interface to identify which of the interchangeable components is in use at any time. Calibration data for each of the interchangeable components may be stored in the apparatus, for example in the computer 142.

Although the invention has been described with reference to specific examples, it will be appreciated by those skilled in the art that the invention may be embodied in many other forms.

1. An apparatus for optical coherence tomography and metrology of an eye,
the apparatus being switchable between an anterior chamber mode and a posterior chamber mode, wherein in the anterior chamber mode the apparatus is configured to provide:
tomographic imaging including metrology of one or more of corneal thickness, anterior chamber depth, lens thickness and axial length of the eye;
wavefront sensing measurements; and
metrology of one or more optical or biological properties selected from the group comprising reflection based corneal topography, tear film breakup and en-face photography or videography;
and wherein in the posterior chamber mode the apparatus is configured to provide tomographic imaging of the retina of the eye.

2. An apparatus for optical coherence tomography and metrology of an eye,
the apparatus being switchable between an anterior chamber mode and a posterior chamber mode, wherein in the anterior chamber mode the apparatus is configured to provide:
tomographic imaging including metrology of one or more of corneal thickness, anterior chamber depth, lens thickness and axial length of the eye;
reflection based corneal topography; and
metrology of one or more optical or biological properties selected from the group comprising wavefront sensing measurements, tear film breakup and en-face photography or videography;
and wherein in the posterior chamber mode the apparatus is configured to provide tomographic imaging of the retina of the eye.

3. The apparatus according to claim 1, comprising an optical relay having one or more electrically actuatable elements for switching the apparatus between the anterior chamber mode and the posterior chamber mode.

4. The apparatus according to claim 3, wherein the one or more electrically actuatable elements comprises a movable element of a zoom lens.

5. The apparatus according to claim 4, wherein the zoom lens has a focal length in the range of 30 to 150 mm, more preferably in the range of 60 to 80 mm and most preferably around 72 mm when the apparatus is in the anterior chamber mode.

6. The apparatus according to claim 4, wherein the zoom lens has a focal length in the range of 15 to 75 mm, more preferably in the range of 30 to 40 mm and most preferably around 36 mm when the apparatus is in the posterior chamber mode.

7. The apparatus according to claim 1, wherein the optical relay includes a lens assembly having a fixed focal length in the range of 30 to 150 mm, more preferably in the range of 60 to 80 mm and most preferably around 72 mm.

8. The apparatus according to claim 3, wherein the one or more electrically actuatable elements comprises one or more lenses for insertion into the optical path when switching to the posterior chamber mode.

9. The apparatus according to claim 8, wherein the one or more lenses comprises a lens pair.

10. The apparatus according to claim 9, wherein the separation between the lenses of the lens pair is adjustable to suit the prescription of the eye.

11. The apparatus according to claim 1, comprising manually interchangeable components for switching the apparatus between the anterior chamber mode and the posterior chamber mode, wherein the manually interchangeable components have electrical interfaces that connect to the apparatus at a registration point.

12. The apparatus according to claim 11, wherein the registration point is achieved with a magnetic ramp and lock.

13. The apparatus according to claim 11, comprising means for identifying the manually interchangeable component currently in use.

14. The apparatus according to claim 11, comprising stored calibration data for each of the manually interchangeable components.

15. The apparatus according to claim 1, wherein the apparatus is configured to provide reflection based corneal topography when in the anterior chamber mode.

16. The apparatus according to claim 15, wherein the apparatus is configured to provide metrology of tear film breakup and en-face photography or videography when in the anterior chamber mode.

17. The apparatus according to claim 2, wherein the apparatus is configured to provide wavefront sensing measurements when in the anterior chamber mode.

18. The apparatus according to claim 17, wherein the apparatus is configured to provide metrology of tear film breakup and en-face photography or videography when in the anterior chamber mode.

19. An article of manufacture comprising a non-transitory computer usable medium having a computer readable program code configured to operate the apparatus according to claim 1.
